(19) United States
(12) Patent Application Publication
(10) Pub. No.: US 2023/0416415 A1
(43) Pub. Date: Dec. 28, 2023

(54) LOW METHOXYL, HIGHLY CALCIUM-REACTIVE PECTIN AND PROCESS FOR ITS PREPARATION

(71) Applicant: Herbstreith & Fox GmbH & Co. KG Pektin-Fabriken, Neuenbürg (DE)

(72) Inventor: Gerhard F. Fox, Neuenbürg (DE)

(21) Appl. No.: 18/247,726

(22) PCT Filed: Sep. 30, 2021
(86) PCT No.: PCT/EP2021/076928

§ 371 Date: Apr. 3, 2023

(30) Foreign Application Priority Data
Oct. 2, 2020 (DE) 10 2020 125 815.1

(51) Int. Cl. C08B 37/00; C12N 9/18; C12P 19/04

(52) U.S. Cl. CPC C08B 37/0048; C12N 9/18; C12P 19/04; C12Y 301/01011

(57) ABSTRACT

The present invention relates to a method for the preparation of a low methoxyl, highly-calcium-reactive pectin and to a low methoxyl, highly-calcium-reactive pectin obtainable by said process. In addition, the present invention relates to the use of the low methoxyl pectin according to the invention for the production of food and non-food products. Furthermore, it is an object of the present invention to provide a product or a stabilized aqueous system prepared using the low methoxyl pectin according to the invention.

The present invention relates to a process for the preparation of a low methoxyl, highly-calcium-reactive pectin and to a low methoxyl, highly-calcium-reactive pectin obtainable by said process. In addition, the present invention relates to the use of the low methoxyl pectin according to the invention for the production of products of the food and non-food area. Furthermore, an object of the present invention is a product or a stabilized aqueous system prepared by using the low methoxyl pectin according to the invention.

### BACKGROUND OF THE INVENTION

Pectins are plant polysaccharides, more specifically polyuronides, substantially consisting of α-1,4-glycosidically bonded D-galacturonic acid units. Low methoxyl pectins are known in the art. They have a degree of esterification of less than 50%. Low-ester pectins are used in particular as gelling or thickening agents for the production of liquid or gel-like food products. In addition to the food sector, the pharmaceutical sector is becoming increasingly important as a field of application for pectins.

For gelation, low methoxyl pectins require polyvalent cations which form chain associates with the carboxyl groups of their galacturonic acid building blocks linked by axial-axial bonding, which is also known in the literature as the egg box model. In food products, calcium ions are commonly used here as polyvalent cations. The firmness of the gels depends not only on the structure of the pectin but also on numerous other factors such as the content of low methoxyl pectin, the calcium ion concentration, the content of soluble solids and the pH value.

On the structural side, numerous parameters play a role in the gel formation using pectin, such as its molecular weight, its content of galacturonic acids, its degree of esterification and also its distribution of ester groups (blockwise vs. random).

As part of the production of a food preparation, in particular one containing fruit, a suitable amount of a calcium salt is added to the respective low methoxyl pectin—typically a low methoxyl, amidated or non-amidated pectin. Alternatively, a specially standardized low methoxyl pectin may be provided, i.e. a dry blend containing the pectin and a calcium salt.

One problem is that the dosage of the calcium salt depends both on its calcium content and on the respective application. When selecting the appropriate amount of calcium salt, the final soluble solids content and other properties of the preparation to be manufactured, in particular the fruit-containing preparation, and of the food mix product to be manufactured using this preparation are of particular importance. For example, the type of fruit used, the addition of milk or milk alternatives and the pH value must be taken into account.

Another disadvantage of the use of low methoxyl, amidated or non-amidated pectins as thickeners with the addition of a calcium salt is that various problems can arise, particularly in the production of preparations which have a relatively low soluble solids content, due to the solubility of the calcium salt used in each case. For example, when using readily soluble calcium salts, e.g. calcium chloride or calcium lactate, a rapid reaction is usually observed due to the high calcium reactivity of low methoxyl pectins. This can lead to undesirable pre-gelation up to precipitation of the pectin as a non-gelling calcium pectinate and subsequently to undesirable texture properties up to syneresis of the preparation. In contrast, a major problem when using poorly soluble calcium salts, e.g. calcium citrate or calcium phosphate, is that the dosed amount of calcium salt is often not sufficient for satisfactory gelation due to poor solubility.

A further disadvantage of the use of low methoxyl, amidated and non-amidated pectins is that the calcium salt intended in each case has to be stated in the list of ingredients of the respective food mix product, which lengthens the list of ingredients and thus makes it less consumer-friendly. In addition, the organic certification for a food mix product, which is becoming increasingly important for consumers, is only possible if the calcium salt used is also organic. Thus, the choice of calcium salt—in addition to its solubility properties—is further restricted by this additional criterion, which may have to be met. A major disadvantage of amidated pectins is that they are not approved per se for use in organic products.

Alternatively, modified and unmodified starches can be used in the prior art as thickeners for yogurt fruit preparations. Modified starches are food additives that are subject to mandatory E-numbering. This labeling requirement does not apply to non-modified starches. Consumers regularly assume that additives labeled with an E-number are not of natural origin. Therefore, they have a low consumer acceptance.

A particular disadvantage of the use of starch, especially non-modified starch (no E number), as a thickener—especially for the production of fruit-containing preparations—is that a comparatively high dosage is required. On the one hand, this has a negative effect on resource efficiency. On the other hand, there is an unfavorable loss of fruit flavor. A further disadvantage is that the use of starch has an unfavorable effect on flavor release, i.e. it is associated with relatively poor flavor release. As a result, the addition of flavoring agents is regularly required, which lengthens the ingredient list of the respective food mix product and usually further decreases consumer acceptance. In addition, starch as a thickening agent has a relatively high cooking viscosity, so that any fruit pieces that may be present are undesirably crushed or turn into puree during the cooking process.

Furthermore, the preparations described in the prior art, in particular those containing fruit, which are intended for use in dairy products, usually have their pH adjusted to the natural pH of the respective dairy product. The pH of such fruit preparations is usually adjusted by means of a pH regulator, such as sodium citrate. This may require a further working and/or measuring step. In addition, the list of ingredients is extended by the respective pH regulator, which may also have to be approved for use in organic products.

Overall, the gelling or thickening agents described in the prior art, which are used to produce pumpable, in particular fruit-containing, preparations with a relatively low soluble solids content, i.e. in the range from 10° Brix to 45° Brix, must be considered unsatisfactory, in particular from a technological, economic and ecological point of view.

The production of high-quality low methoxyl pectins is technically demanding. On the one hand, the pectin can be de-esterified by treatment with acids or lyse. However, this treatment also leads to hydrolytic cleavage of the polygalacturonic acid chains, which has a detrimental effect on the pectin quality. In addition, de-esterification in an acidic environment is associated with a long incubation period. Moreover, the resulting low methoxyl pectins often acquire a yellow tinge. In the prior art, enzymatic pectin de-esterification using a pectin methyl esterase is also common. As an enzymatic reaction, it is particularly sensitive to reaction conditions, such as pH or the presence of calcium ions.

DE 601 22 522 T2 concerns pectins with a low degree of methoxylation and teaches in paragraph [0022] a low methoxyl pectin with a degree of esterification of about 20 to 50%. According to Table A ([0158]), a defined amount of calcium (in the form of a CaCl₂) solution) must be added when using this pectin to thicken a synthetic beverage.

WO 99/37685 concerns a pectin for pasty materials and teaches in claim 1 a pectin with a molecular weight of about 30 to 40 kDa and an esterification degree of less than 20%. De-esterification under the alkaline conditions taught also results in pectin hydrolysis (molecular weight reduction; see claim 7 and example on page 8, lines 21 to 23).

U.S. Pat. No. 3,622,559 relates to pectins with high breaking strength teaches in claim 1 a process for the preparation of a low methoxyl pectin with a degree of esterification of between 7 to 8.5% in which the de-esterification takes place in an acidic environment (0.7-1.5 N HCl) over a very long period of at least 24 hours (column 3, lines 3 to 7).

The objective of the present invention thus is to overcome the above-mentioned and further disadvantages of the prior art.

### SUMMARY OF THE INVENTION

In a first aspect of the present invention, the objective posed is solved by a process for the preparation of low methoxyl pectin, the process comprising the following steps:
(a) Providing a pectin-containing starting biomass material that includes an insoluble fiber component and an insoluble protopectin component;
(b) Extraction of the pectin-containing starting biomass material with an aqueous medium at an acidic pH under conditions where at least a portion of the pectin content is extracted;
(c) Separation of the pectin from the treated starting biomass material and at least partial removal of divalent cations from the pectin extract;
(d) Concentrating the pectin extract from step (c) so that the pectin extract has a content of pectin-containing material as dry matter that is in the range of 0.5 to 10 wt %;
(e) Contacting the concentrated pectin extract from step (d) in an aqueous solution with a pectin methyl esterase (E.C. 3.1.1.11) at a pH of 3.8 to 4.5;
(f) Allow the pectin methylesterase to de-esterify the pectin to produce a de-esterified pectin by incubating the aqueous suspension from step (e);
(g) Terminating the de-esterification when the de-esterified pectin has a degree of esterification of from 10 to 34% and advantageously from 10 to 28.0%, and
(h) Obtaining low methoxyl pectin therefrom,

wherein in step (f) a further adjustment of the pH value is carried out by adding a pH-value-increasing substance or substance mixture.

The production process according to the invention leads to low methoxyl pectin with a high calcium reactivity. This low methoxyl, highly calcium-reactive pectin of the invention is also referred to synonymously below as “low methoxyl” pectin.

The low methoxyl pectin according to the invention is a non-amidated pectin. Neither an ammonolysis of high methoxyl pectin nor a reaction of the low methoxyl pectin with ammonia takes place in the production process according to the present invention. The low methoxyl non-amidated pectin according to the present invention is also referred to hereinafter as “low methoxyl pectin”.

Surprisingly, it was found that the low methoxyl pectin claimed here exhibits optimal gelling properties, and depending on the proportion of dry matter, even without the addition of a typically provided calcium salt, such as calcium lactate or calcium citrate. This surprising result is directly attributable to very high calcium reactivity and the very good solubility of the low methoxyl pectin claimed here, especially in water.

The pH adjustment in the de-esterification step maintains an optimum pH level for pectin methyl esterase, and thus esterification levels of 34% or less, and advantageously 28.0% or less, can be achieved while maintaining a pumpable mass.

In addition, the pH adjustment gently de-esterifies the pectin. There is no strong acidification or basification. The pectin backbone thus retains its structural integrity.

The process according to the invention is simple and inexpensive to carry out using proven substances and can therefore be easily implemented in existing industrial processes and devices for pectin processing.

The low methoxyl pectin produced herewith is thus able to ensure sufficient gelation without the addition of calcium ions, even in stabilized aqueous systems (such as fruit preparations) which have only a low soluble solids content of 30 to 45%. In addition, the low methoxyl pectin produced herewith can also be used for gelation in stabilized aqueous systems with an even lower soluble solids content of 10 to 30% (corresponding to 10 to 30° Brix). An example of this is fruit compote.

Advantageously, the addition of a typically provided calcium salt, such as calcium lactate or calcium citrate, can thus be dispensed with in fruit preparations. This result is due to the high calcium reactivity of the low methoxyl according to the invention. Due to this high calcium reactivity, the fruit's own content of calcium ions is sufficient to achieve the desired gelation.

It was found that the gelled foods produced with the pectin produced according to the invention have a creamy, spreadable texture. In addition, they exhibit only a very low tendency to syneresis, and no disturbing phase separation is observed.

Vegetable processing residues such as apple pomace or citrus pomace can be used as raw material in the production process according to the invention. These processing residues are inexpensive, are available in sufficient quantities and provide a sustainable and ecologically sensible source for the pectin according to the invention.

Low methoxyl pectins are established and accepted in the food industry, so that corresponding compositions can be used immediately and also internationally without lengthy approval procedures.

### DETAILED DESCRIPTION OF THE INVENTION

In the first aspect, the invention relates to a process for the preparation of a low methoxyl highly-calcium-reactive pectin comprising the preparation steps (a) to (g).

The starting material used in this process is a pectin-containing starting biomass material that includes an insoluble fiber component and an insoluble protopectin component. The pectin-containing starting biomass material is a plant material comprising primary cell walls. The primary wall of plant cells is composed of pectins, cellulose, hemicellulose and proteins. Accordingly, plants or plant parts rich in primary walls are the typical starting material for pectin isolation. These include, for example, apple pomace, citrus pomace or sugar beet pulp.

The extraction in aqueous suspension in the acidic pH environment according to step (b) disintegrates the starting biomass material. Incubation in the aqueous liquid as a suspension causes the starting biomass material to swell and become permeable. This allows penetration of the acid into the plant material, where it can convert the protopectin into water-soluble pectin. This water-soluble pectin can now be dissolved out of the permeabilized biomass material while retaining the pectin structure and is thus present in the aqueous liquid as extractable pectin.

In step (c), the separation of the pectin dissolved in the liquid from the solid starting biomass material treated according to step (b) is then carried out. This is typically done via solid-liquid separation. Step (c) also includes at least partial removal of divalent cations from the extract, such as magnesium and especially calcium.

In step (d), the pectin extract is concentrated so that it has a content of pectin-containing material as dry matter which is in the range of 0.5 to 10 wt % and preferably in the range of 6 to 8 wt %. It turned out that pectin methyl esterase exhibits satisfactory de-esterification only at such an adjusted pectin dry matter.

The de-esterification of pectin is achieved by contacting the concentrated pectin extract in aqueous suspension with a pectin methyl esterase (E.C. 3.1.1.11) at a pH of 3.8 to 4.5 according to step (e) and subsequent incubation under suitable reaction conditions according to step (f).

The enzymatic de-esterification reaction is terminated according to step (g) as soon as the desired degree of esterification is reached. This can be done, for example, by precipitation of the pectin or denaturation of the enzyme.

In the process according to the invention, it is crucial that the pH is adjusted during the incubation of the de-esterification reaction mixture according to step (f). This is done by adding a pH-value-increasing substance or substance mixture.

In the process according to the invention, biocatalytic, i.e. enzymatic, de-esterification takes place. Due to the setting of controlled reaction conditions, such as pH or calcium ion concentration, gentle de-esterification can be achieved with esterification degrees of 34% or less and advantageously of 28.0% or less. Additional chemical de-esterification (i.e. by exposure to acids, bases or ammonia) is not necessary.

In a preferred embodiment, the pectin-containing starting biomass material is a fruit selected from the group consisting of citrus, apple, sugar beet, infructescences of sunflower, rosehip, quince, apricot, cherry, carrot, and mixtures thereof. In a preferred form, it is a fruit component of such a fruit or the residue of such a fruit as a result of a processing operation. This is, for example, the process of juice extraction (as in apple or citrus) or the process of sugar extraction (as in sugar beet).

In a second aspect, the invention provides a process for producing low methoxyl pectin using a pectin starting material as the starting material. In contrast to the pectin-containing starting biomass material of the first aspect of the invention, the pectin in the pectin starting material is already present as extracted and thus isolated pectin in solid form. Thus, a starting material already extracted and therewith usually present as high methoxyl pectin can also be advantageously modified by the esterification process according to the invention, in which it is converted into a low methoxyl highly-calcium-reactive pectin.

The process for producing low methoxyl pectin according to the second aspect comprises the following steps:
(b1) Providing a pectin starting material in a dried state;
(c1) Preparing an aqueous solution of the pectin starting material from step (b1) such that the pectin-containing solution has a content of pectin-containing material as dry matter that is in the range of 0.5-10 wt %;
(e) Contacting the concentrated pectin-containing solution from step (c1) in an aqueous solution with a pectin methyl esterase (E.C. 3.1.1.11) at a pH of 3.8 to 4.5;
(f) Allow the pectin methylesterase to de-esterify the pectin to produce a de-esterified pectin by incubating the aqueous solution from step (e);
(g) Terminating the de-esterification when the de-esterified pectin has a degree of esterification of from 10 to 34% and advantageously from 10 to 28.0%, and
(h) Obtaining low methoxyl pectin therefrom,

wherein in step (f) a further adjustment of the pH value is carried out by adding a pH-value-increasing substance or substance mixture.

As described above, in step (b1) a pectin starting material in dried and thus solid form is provided as starting material.

According to step (c1) of the process, the pectin starting material is dissolved in an aqueous liquid to produce an aqueous solution.

In this process, the pectin is dissolved so that it has a content of pectin-containing material as dry matter that is in the range of 0.5-10 wt %. It turned out that pectin methyl esterase exhibits satisfactory de-esterification only at such an adjusted pectin dry matter.

The de-esterification of pectin is achieved by contacting the concentrated pectin extract in aqueous suspension with a pectin methyl esterase (E.C. 3.1.1.11) at a pH of 3.8 to 4.5 according to step (e) and subsequent incubation under suitable reaction conditions according to step (f).

The enzymatic de-esterification reaction is terminated according to step (g) as soon as the desired degree of esterification is reached. This can be done, for example, by precipitation of the pectin or denaturation of the enzyme.

Analogous to the first aspect of the invention, it is also crucial in the method according to the invention according to aspect 2 that the pH is adjusted during the incubation of the de-esterification reaction mixture according to step (f). This is done by adding a pH-value-increasing substance or substance mixture.

In a preferred embodiment, the pectin starting material is obtained from a fruit selected from the group consisting of citrus, apple, sugar beet, infructescences of sunflower, rosehip, quince, apricot, cherry, carrot, and mixtures thereof.

In a particular embodiment, the pectin starting material is obtained from citrus or apple raw materials and is thus advantageously of natural origin. Usually, vegetable processing residues such as citrus or apple pomace are used for the isolation of this pectin starting material. These are available in sufficient quantities and provide a sustainable and ecologically sound source for the required natural starting materials. Among other things, citrus pectin with various degrees of esterification can be obtained from citrus pomace. Accordingly, apple pectin with different degrees of esterification can be obtained from apple pomace.

In the process according to the invention according to the first aspect, the pectin-containing starting biomass material may be in a moist, non-dried or dried state. Thus, during juice production, the pomace is obtained as a moist mass. This moist pomace can initially be used fresh as moist biomass in the process as pectin-containing starting biomass material. Alternatively, the pomace can also be frozen and the process then carried out starting from the frozen pomace, with the frozen pomace then being thawed in a first step.

In a particularly preferred embodiment, the pectin-containing starting biomass material is a citrus pomace or apple pomace. These pomaces are available in sufficient quantities and provide a sustainable and ecologically sound source for the required natural starting materials. Among other things, citrus pectin with various degrees of esterification can be obtained from citrus pomace. Accordingly, apple pectin with different degrees of esterification can be obtained from apple pomace.

The citrus pulp of a wide variety of citrus fruits can be used for the isolation of citrus pectin. In a non-restrictive manner, examples are listed here: Mandarin (Citrus reticulata), Clementine (Citrus x aurantium Clementine group, Syn.: Citrus clementina), Satsuma (Citrus xaurantium Satsuma group, Syn.: Citrus unshiu), Mangshan (Citrus mangshanensis), Orange (Citrus xaurantium orange group, Syn.: Citrus sinensis), Bitter Orange (Citrus xaurantium bitter orange group), Bergamot (Citrus xlimon bergamot group, Syn.: Citrus bergamia), grapefruit (Citrus maxima), grapefruit (Citrus xaurantium grapefruit group, syn.: Citrus paradisi) pomelo (Citrus xaurantium pomelo group), true lime (Citrus xaurantiifolia), common lime (Citrus xaurantiifolia, syn. Citrus latifolia), kaffir lime (Citrus hystrix), Rangpur lime (Citrus xjambhiri), lemon (Citrus xlimon lemon group), citron (Citrus medica) and kumquats (Citrus japonica, Syn.: Fortunella). Preferred among these are orange (Citrus xaurantium orange group, syn.: Citrus sinensis) and lemon (Citrus xlimon lemon group).

Acidic Extraction According to Step (b)

The extraction in step (b) of the process serves to remove at least part of the pectin from the cell association by converting a partial fraction of the protopectin into soluble pectin.

The extraction conditions in step (b) are such that the pectin extracted during the extraction process is a high methoxyl pectin with high gelling strength and good viscosifying ability. It is therefore also referred to as “high quality pectin” in the context of the present application.

The acidic extraction according to step (b) is a full pectin extraction in the sense that the pectin brought into solution is thereafter separated from the fiber material by solid-liquid separation according to step (c).

The high quality pectin resulting from the partial extraction is a high methoxyl pectin. A high methoxyl pectin according to the invention is a pectin with a degree of esterification of at least 50%. The degree of esterification describes the percentage degree of carboxylic groups in the galacturonic acid units of the pectin which are present in esterified form, e. g. as methyl esters. The degree of esterification can be determined by the method according to JECFA (monograph 19-2016, Joint FAO/WHO Expert Committee on Food Additives).

According to an advantageous embodiment, the high-quality pectin, which is preferably a high methoxyl soluble citrus pectin or apple pectin, has a degree of esterification of from 50 to 80%, preferably from 60 to 80%, particularly preferably from 70 to 80%, and especially preferably from 72% to 75%. For example, the degree of esterification of the high methoxyl soluble pectin, which is preferably a high methoxyl soluble citrus pectin or apple pectin, may be 70%, 71%, 72%, 73%, 74%, 75%, 76%, 77%, 78%, 79% or 80%.

According to one embodiment, the high-quality pectin, which is preferably a high methoxyl soluble citrus pectin, has a viscosity, measured in mPas, of from 400 to 1000 mPas, preferably from 500 to 1000 mPas, more preferably from 600 to 1000 mPas, and especially preferably from 700 to 1000 mPas. The viscosity of the high-quality pectin which is preferably a high methoxyl soluble citrus pectin, may be 550, 600, 650, 700, 750, 800, 850, 900 or 950 mPas.

According to one embodiment, the high-quality pectin, which is preferably a high methoxyl soluble citrus pectin, has a gelling strength, measured in ° SAG, of from 150 to 260° SAG, preferably from 200 to 260° SAG, particularly preferably from 220 to 260° SAG, and especially preferably from 230 to 245° SAG. For example, the gelling power of the high methoxyl soluble pectin, which is preferably a high methoxyl soluble citrus pectin, may be 160, 170, 180, 190, 200, 210, 220, 230, 240 or 250° SAG.

According to one embodiment, the high-quality pectin, which is preferably a high methoxyl soluble apple pectin, has a gelling strength, measured in ° SAG, of from 150 to 250° SAG, preferably from 170 to 240° SAG, particularly preferably from 180 to 220° SAG, and especially preferably from 190 to 200° SAG. For example, the gelling power of the high methoxyl soluble pectin, which is preferably a high methoxyl soluble apple pectin, may be 160, 170, 180, 190, 191, 192, 193, 194, 195, 196, 197, 198, 199, 200, 210, 220, 230 or 240° SAG.

The pectin-containing starting biomass material is present as an aqueous suspension during disintegration. A suspension according to the invention is a heterogeneous mixture of a liquid and solids (raw material particles) finely distributed therein. Since the suspension tends to sedimentation and separation of phases, the particles are suitably kept in suspension by shaking or stirring. That is, there is no dispersion, which would mean that the particles are mechanically comminuted (shearing) so as to be finely dispersed.

To achieve an acidic pH value the person skilled in the art may employ all acids or acidic buffering solutions that are known to him. For example, an organic acid can be used that acts as a calcium chelator and can thus bind excess calcium ions. Examples of such a chelating acid are citric acid, gluconic acid or oxalic acid.

Alternatively, or in combination, a mineral acid may be used. Some examples are sulfuric acid, hydrochloric acid, nitric acid or sulfurous acid. Preferably, nitric acid or sulfuric acid is employed.

In the extraction of step (b) of the method, the pH value of the suspension is between pH=0.5 and pH=4.0, preferably between pH=1.0 and pH=3.5 and particularly preferably between pH=1.0 and pH=3.0. For example, the acidic disintegration can be performed at a pH of 1.5, 1.6, 1.7, 1.8, 1.9, 2.0, 2.1, 2.2, 2.3, 2.4, 2.5, 2.6, 2.7, 2.8 or 2.9.

In the extraction the incubation takes place at a temperature of between 60° C. and 95° C., preferably of between 70° C. and 90° C., and particularly preferably of between 75° C. and 85° C. For example, the extraction can be performed at a temperature of 76° C., 77° C., 78° C., 79° C., 80° C., 81° C., 82° C., 83° C. or 84° C.

In the extraction the aqueous suspension suitably has a dry matter of between 0.5 wt % and 5 wt % preferably of between 1 wt % and 4 wt %, and particularly preferably of between 1.5 wt % and 3 wt %. For example, the dry matter may be 1.6, 1.7, 1.8, 1.9, 2.0, 2.1, 2.2, 2.3, 2.4, 2.5, 2.6, 2.7, 2.8 or 2.9 wt % for the acidic disintegration.

During the disintegration the aqueous suspension is suitably set in motion by the application of force, e.g. stirred or shaken. This is preferably done in a continuous manner so that the particles in the suspension are kept in suspension.

Separation and Removal of Cations According to Step (c)

In step (c) of the process, the extracted pectin dissolved in the liquid is separated from the treated biomass and thus made available for further processing. This separation is carried out as a single-stage or multi-stage solid-liquid separation.

Advantageously, the disintegrated material is subjected to a multi-stage solid-liquid-separation. Preferably, the first separation of particles is done by means of decanters and the second one by means of separators. Here, based on the aqueous fiber suspension the solid is separated independently of the particle size from the liquid, contrary to a classification process.

Optionally, larger particles can also be separated in step (c) This is preferably done by a classification process. In the context of the present invention, a classification process is understood to mean the separation of a disperse solid mixture into fractions according to particle size. In the simplest case, two fractions are produced here, but two or three particle fractions with a defined particle distribution can also be produced as part of the classification process. The classic process here is sieving.

Here, particularly advantageous is the separation of particles with a particle size of more than 500 μm, preferably more than 400 μm and most preferably more than 350 μm. The separation is advantageously carried out with a straining machine or a sieving drum. This removes both coarse particulate impurities from the raw material and insufficiently disintegrated material. The need to perform this optional separation step depends on the firmness of the fiber material to be disintegrated. While it is regularly necessary for citrus fibers, apple fibers disintegrate into fine fibers during the acid hydrolysis step, so that this separation step can be regularly omitted.

Alternatively, wet sieving can be used as the sieving process.

The skilled person is familiar with numerous sieving machines for carrying out classification processes, which he will select according to the fiber particle size present and the fact that a moist or wet material is present. Examples of sieving machines include cantilever sieving machines, elliptical sieving machines, eccentric sieving machines, linear sieving machines, throw sieving machines, planar sieving machines, beater sieving machines, air jet sieving machines, and eddy current machines.

The classification process in step (c) can be carried out during the single or multi-stage separation of the disintegrated material from the aqueous liquid, before this separation, or after the separation from the aqueous liquid.

In addition to pectin separation, step (c) also includes removal of divalent cations. Here, at least a part of the divalent cations is removed, preferably a complete removal of divalent cations takes place. By a removal according to the invention is meant both the removal of the cations from the solution and the functional removal as performed by a chelator. In a functional removal, the divalent cation present in the solution can no longer form a complex with the pectin due to its chelation. These cations are preferably magnesium or calcium and especially calcium. The removal of the divalent ions in step (c) can be carried out in various ways.

Thus, in one embodiment, a cation exchanger material may be used that is either implemented in a batch process or a column process.

For example, an inorganic substance such as zeolite can be used as the cation exchanger material. Preferably, however, an organic substance and in this case a synthetic resin-based cation exchanger is used as the material. Cation exchangers based on organic bases, for example, can be used as cation exchanger materials. These are weak bases and can carry tertiary amine groups as functional groups. Examples include the cation exchanger resins Purolite A 100 Plus and Purolite PPC 150 S (Lenntech, Delfgauw, Netherlands).

In an alternative embodiment, a chelator may be used here to remove the divalent ions, i.e., an inorganic or organic substance capable of fixing the divalent cations in stable, ring-shaped complexes (the so-called chelates).

In another embodiment, the removal of the divalent ions may be accomplished by precipitation as a poorly soluble salt. For example, calcium ions can be precipitated as a poorly soluble calcium oxalate by addition of a soluble oxalate salt (such as sodium oxalate).

Divalent cation removal can be performed in step (c) either before pectin separation, during pectin separation, or after pectin separation.

Thus, the cation exchanger can be added as particulate material prior to pectin separation, so that separation from the solid cation exchanger is also achieved by pectin separation as solid-liquid separation.

Concentrating the Pectin Extract of Step (d)

In step (d), the pectin extract obtained in step (c) is concentrated to give as a result a pectin extract having a content of pectin-containing material as dry matter ranging from 0.5 to 10 wt %. Numerous methods for concentrating aqueous solutions are known to the skilled person. Concentration can be carried out, for example, by evaporating the water as solvent, preferably under vacuum to reduce the boiling temperature. Other examples include ultrafiltration, reverse osmosis on semipermeable membranes and precipitation of the pectin by an organic solvent with subsequent absorption in a reduced volume of solvent.

Enzymatic De-Esterification According to Step (e)

According to the invention, in the de-esterification according to step (e), at least one pectin methyl esterase (EC 3.1.1.11) is added to the aqueous pectin solution.

The pectin methylesterase hydrolyzes the methyl esters of the galacturonic acid groups in the pectin to form poly-galacturonic acid and methanol. The resulting low methoxyl pectins can form a gel in the presence of polyvalent cations even without sugar and can also be used in a wide pH range.

A pectin methylesterase (abbreviation: PME, EC 3.1.1.11, also: pectin demethoxylase, pectin methoxylase) is a commonly found enzyme in the cell wall in all higher plants and some bacteria and fungi, which cleaves the methyl esters of pectins, forming poly-galacturonic acid and releasing methanol. PME has been isolated in many isoforms, all of which can be used for enzymatic de-esterification according to the invention. Thus, PME has been isolated in many isoforms from plant pathogenic fungi such as Aspergillus foetidus and Phytophthora infestans as well as from higher plants, e.g. tomatoes, potatoes and oranges. Fungal PMEs exhibit optimal activity between pH 2.5 and 5.5, whereas plant PMEs exhibit pH optima between pH 5 and 8. The relative molecular mass is between 33,000 and 45,000. The enzyme is present as a monomer and is glycosylated. The KM value ranges from 11 to 40 mM pectin in fungal PME and from 4-22 mM pectin in plant PME. The commercially available preparations of PME are obtained either from the supernatants of fungal mycelial cultures or, in the case of plants, from fruits (peels of oranges and lemons, tomatoes). The preferred pectin methylesterases have a pH optimum between 2 and 5 and a temperature optimum at 30 to 50° C., although depending on the enzyme, appreciable enzyme activity can be observed from as low as 15° C.

The following table shows some examples of commercially available PME with their reaction optima:

| Product name | Manufacturer | Optima |
|---|---|---|
| Rapidase PEP | DSM | pH = 4-5; T = 50° C. |
| Pectinase 872 L | Biocatalysts | pH = 4-5; T = 30-50° C. |
| Pectinesterase | Erbslöh | pH = 4-5; T = 30-50° C. |
| 1508/14 | | |

In the enzymatic de-esterification, the aqueous solution contains the pectin methylesterase in a total activity of from 100 to 10,000 units/L, advantageously from 500 to 5000 units/L, and particularly advantageously from 1000 to 2500 units/L.

In the enzymatic de-esterification, the incubation with the at least one pectin methyl esterase in the aqueous solution is carried out for a period of 10 to 20 hours and preferably 12 to 18 hours. The incubation period in this case may be, for example, 11, 12, 13, 14, 15, 16, 17, 18 or 19 hours. In this context, the reaction time for the de-esterification reaction depends in particular on the pectin concentration, the respective PME isoform, the addition rates, the solvent or solvent mixture, the selected pH value or pH value progression and the de-esterification temperature.

In the enzymatic de-esterification, the incubation with the at least one pectin methyl esterase in the aqueous solution takes place at a temperature of between 10° C. and 70° C., preferably of between 20° C. and 60° C. and particularly preferably of between 30° C. and 50° C. In this regard, the incubation temperature may be, for example, 20.5° C., 21.0° C., 21.5° C., 22.0° C., 22.5° C., 23.0° C., 23.5° C., 24.0° C., 24.5° C., 25.5° C., 26.0° C., 26.5° C., 27.0° C., 27.5° C., 28.0° C., 28, 5° C., 29.0° C., 29.5° C., 30.5° C., 31.0° C., 31.5° C., 32.0° C., 32.5° C., 33.0° C., 33.5° C., 34.0° C., 34.5° C., 35.5° C., 36.0° C., 36.5° C., 37.0° C., 37.5° C., 38.0° C., 38.5° C., 39.0° C., 39.5° C., 40.0° C., 40.5° C., 41.0° C., 41.5° C., 42.0° C., 42.5° C., 43.0° C., 43.5° C., 44.0° C., 44.5° C., 45.0° C., 45.5° C., 46.0° C., 46.5° C., 47.0° C., 47.5° C., 48.0° C., 48.5° C., 49.0° C., 49.5° C., 50, 0° C., 50.5° C., 51.0° C., 51.5° C., 52.0° C., 52.5° C., 53.0° C., 53.5° C., 54.0° C., 54.5° C., 55.0° C., 55.5° C., 56.0° C., 56.5° C., 57.0° C., 57.5° C., 58.0° C., 58.5° C., 59.0° C. or 59.5° C. In the enzymatic de-esterification reaction, the choice of temperature depends in particular on the activity range of the respective enzyme isoform or enzyme mixture.

In the enzymatic de-esterification, the incubation with the at least one pectin methyl esterase is carried out in the aqueous solution at an initial pH of from 3.8 to 4.5, preferably from 4.0 to 4.3 and particularly preferably from 4.1 to 4.2. The pH in this case can be, for example, 3.9, 4.0, 4.1, 4.2, 4.3 or 4.4. In this context, the choice of the pH range or pH value depends in particular on the activity range of the respective enzyme or enzyme mixture, the pH value having to be at least 3.8, and preferably at least 4.0, in order to ensure efficient de-esterification while preventing in situ gelation of the de-esterified pectin.

In the enzymatic de-esterification, the dry matter in the aqueous suspension is from 0.5 wt % to 10 wt %, preferably from 4 wt % to 10 wt %, particularly preferably from 5 wt % to 9 wt %, and especially preferably from 6 wt % to 8 wt %. The dry matter in this case can be, for example, 0.6, 0.7, 0.8, 0.9, 1.0, 1.5, 2.0, 2.5, 3.0, 3.5, 4.0, 4.5, 5.0, 5.5, 6.0, 6.5, 7.0 or 7.5 wt %.

In the de-esterification process, the aqueous solution is suitably set in motion during incubation by the application of force. This can be done by shaking or stirring the solution, taking care that the enzyme does not foam up.

A further optional embodiment of the process provides that the pectin present in step (e) has a calcium ion concentration of less than 0.10 wt %, advantageously less than 0.08 wt %, and in particular less than 0.05 wt %. In this case, the calcium ion concentration of the high methoxyl pectin is reduced in advance, for example using a cation exchanger, to a value which lies in the above-mentioned range. The calcium ion concentration of the high methoxyl pectin in step (e) may, for example, be less than 0.09 wt %, 0.08 wt %, 0.07 wt %, 0.06 wt %, 0.05 wt %, 0.04 wt %, 0.03 wt %, 0.02 wt % or 0.01 wt % as a result of the cation exchanger treatment.

The degree of esterification of pectin in the course of the de-esterification reaction is easy to control and adjust.

In order to achieve a predetermined degree of esterification, the degree of esterification can be determined or controlled during the de-esterification reaction using an analytical method described further below (cf. test method 1.1) at predefined time intervals. Once the predetermined degree of esterification has been reached, the de-esterification reaction can be terminated in a targeted manner by quenching by means of at least one alcohol, i.e. precipitation of the low methoxyl pectin, and/or supply of heat and/or addition of at least one inhibitor, in particular an enzyme inhibitor.

Adjustment of the pH Value in Step (f)

According to the invention, in the process according to one of the preceding claims, the pH is adjusted by adding a pH-value-increasing substance or substance mixture. This addition of a pH-increasing substance proves to be advantageous from a process engineering point of view, because the pH value decreasing due to the enzymatic de-esterification (neutral ester groups are converted into carboxylic acid groups) is thereby increased again in a controlled manner and an optimum pH range is maintained with regard to pectin quality and enzyme activity.

In one embodiment, this adjustment of the pH in step (f) is a stepwise pH increase, which is carried out by discrete addition of a pH-value-increasing substance or substance mixture. This discrete addition can take place in the course of the enzymatic de-esterification as a single addition or as multiple additions, e.g., as two, three, four or five additions.

The discrete addition of the pH-value-increasing substance or substance mixture can take place here as a function of the incubation period or when a specific pH value is reached.

Thus, the discrete addition of the pH-value-increasing substance or mixture of substances can be carried out for an incubation period of 1 to 6 h, preferably from 1.5 to 4 h, and more preferably from 2 to 3 h.

Accordingly, the discrete addition of the pH-value-increasing substance or mixture of substances can take place when a pH of between 3.5 and 4.5 is reached, preferably at a pH of 3.7 to 4.2 and more preferably at a pH of 3.8 to 4.1.

In an alternative embodiment, the adjustment of pH in step (f) is a pH maintenance step performed by continuous addition of a pH-value-increasing substance or mixture of substances.

In a preferred embodiment, the pH-value-increasing substance is a base. This can be added as a single substance or as a mixture of substances, the mixture of substances preferably being an aqueous solution of the corresponding base.

The person skilled in the art can make use of the bases known to him. Preferably, the base is selected from the group consisting of ammonia, sodium hydroxide or potassium hydroxide.

In an alternative embodiment, the pH-value-increasing substance is a slightly acidic or basic salt. This salt can be added as a single substance or as a mixture of substances, the mixture of substances preferably being an aqueous solution of the corresponding salt.

The person skilled in the art can make use of the salts known to him. Preferably, the slightly acidic or basic salt is selected from the group consisting of potassium carbonate, sodium carbonate, sodium hydrogen carbonate and potassium hydrogen carbonate.

In another alternative embodiment, the pH-value-increasing substance mixture is a slightly acidic or basic buffer system.

The person skilled in the art can make use of the buffer systems known to him. Preferably, the slightly acidic or basic buffer system is selected from the group consisting of carbonic acid/carbonate buffer, acetic acid/acetate buffer, phosphate buffer, ammonia buffer, HEPES buffer, PBS buffer and MES buffer.

The low methoxyl pectin obtained by the process can then be suitably isolated from the de-esterification solution according to step (h). This can conveniently be done by precipitation of the pectin with a water-miscible organic solvent.

For this purpose, the de-esterification solution can be concentrated before the precipitation step. This can be done, for example, by membrane filtration or vacuum evaporation. In the case of pectin recovery, it is expedient to concentrate before the de-esterification step. In an alternative embodiment, however, concentration can also take place after de-esterification.

Water-miscible, thermally stable, volatile solvents containing only carbon, hydrogen and oxygen, such as alcohols, ethers, esters, ketones and acetals, are particularly suitable for carrying out the precipitation step according to the invention. Ethanol, n-propanol, isopropanol, methyl ethyl ketone, 1,2-butanediol-1-methyl ether, 1,2-propanediol-1-n-propyl ether or acetone are preferably used.

An organic solvent is referred to herein as “water-miscible” if it is present in a 1:20 (v/v) mixture with water as a single-phase liquid.

In general, solvents that are at least 10% water-miscible, have a boiling point below 100° C. and/or have fewer than 10 carbon atoms are used.

The water-miscible organic solvent as a component of the washing liquid is preferably an alcohol, advantageously selected from the group consisting of methanol, ethanol and isopropanol. In a particularly preferred manner, it is isopropanol.

The pectin can be dehydrated, dried and ground. Dehydration is performed to remove the mass of water prior to the drying step. While any known process can be used for dehydration, the pectin precipitate is preferably treated with alcohol. The water/alcohol phase formed during dehydration is essentially removed by decantation, centrifugation or filtration using known procedures.

Drying is effected by known techniques, e.g. in atmospheric oven or oven at reduced pressure, to a moisture content of less than about 50 wt %, preferably less than about 25 wt %. The drying temperature is maintained below the temperature at which the pectin begins to lose its properties (e.g., in terms of color or molecular weight). Any known milling process can be used to grind the pectin product to the desired particle size. It is particularly preferred that the final product be in dry powder form with a moisture content of about 12 wt % or less. A dry powder form is intended to mean that the product is pourable without substantial baking. The preferred end product is in powder form for ease of use.

The low methoxyl pectin according to the invention can be further mixed with a standardizing agent for use to form a standardized low methoxyl pectin.

A “standardizing agent” in the sense of the invention is defined as an organic uncharged molecule with good water solubility. The standardizing agent serves to standardize the product. The controlled identical manufacturing processes result in pectins with predetermined properties. However, due to raw material-related variations within the pectin composition, these have a certain variation, e.g. with regard to gel firmness or viscosity. The addition of a standardizing agent significantly reduces the range of variation and thus standardizes the pectin. This enables a constant dosage from batch to batch.

Examples of standardizing agents include monosaccharides, oligosaccharides, polysaccharides or sugar alcohols, or combinations thereof.

In the case of the monosaccharide or oligosaccharide, the skilled person can make use of all sugars used in the food industry. The following are examples of the sugars that can be used: Dextrose, sucrose, fructose, invert sugar, isoglucose, mannose, melezitose, maltose, rhamnose, the sugar preferably being sucrose or dextrose.

A standardizing agent such as dextrose or sucrose can be added to the low methoxyl pectin of the invention in a proportion of 20 to 50 wt %, based on the low methoxyl pectin.

The Low Methoxyl Pectin

In a third aspect of the present invention, the objective of the invention is solved by providing a low methoxyl pectin obtainable or obtained by the process according to the invention and having a degree of esterification of from 10 to 34% and advantageously from 10 to 28.0%. For example, the degree of esterification of the low methoxyl pectin may be 11%, 12%, 13%, 14%, 15%, 16%, 17%, 18%, 19%, 20%, 21%, 22%, 23%, 24%, 25%, 26%, 27%, 28.0%, 29%, 30%, 31%, 32% or 33%.

In one embodiment, the low methoxyl pectin according to the invention has a calcium sensitivity of 300 HPE to 3000 HPE. It thus exhibits a very high calcium sensitivity, which allows the pectin according to the invention to be used for preparations without the need for a separate addition of a calcium salt. Here, the amounts of calcium contained in foodstuffs (such as fruit preparations) are sufficient for gelation.

According to the present invention, the term “calcium sensitivity” is understood to be a measure of the firmness of a gel prepared with sucrose in a buffer solution in the presence of a defined calcium ion concentration at pH approx. 3.0 and formed at 22° Brix. Calcium sensitivity is determined after cooling in a water bath at 20° C. for two hours. Calcium sensitivity is determined using the Herbstreith Pectinometer Mark IV. The method used is hereinafter referred to as the calcium sensitivity test, the measured value as the calcium sensitivity, and the units of measurement are HPE (Herbstreith Pectinometer Units, german: Herbstreith Pektinometer Einheiten).

Another embodiment of the low methoxyl pectin provides that it is in powder form, as a liquid, or as a suspension or solution in a solvent selected from the group consisting of water and water-miscible solvents, and mixtures thereof.

“Miscible” means here that two solvents form one phase at least during a reaction, in particular a gelation process, i.e. do not exist as two phases.

The term “water-miscible solvents” includes alcohols, in particular methanol, ethanol, n-propanol and iso-propanol, as well as other organic solvents such as acetone, acetonitrile, methyl acetate, ethyl acetate, and mixtures thereof. The term “alcohols” also includes polyvalent alcohols, i.e. polyols, especially diols and triols. Examples of a diol are ethane-1,2-diol and propane-1,2-diol; an example of a triol is propane-1,2,3-triol.

In yet another embodiment, the low methoxyl pectin has a pH of between 3.0 and 5.5, advantageously between 3.6 and 4.7 and in particular between 3.8 and 4.5. In this pH range, the low methoxyl pectin exhibits its greatest chemical stability. For example, the pH of the low methoxyl pectin can therefore also be 3.6, 3.7, 3.8, 3.9, 4.0, 4.1, 4.2, 4.3, 4.4 or 4.5.

Use of the Pectin

In a fourth aspect, the invention relates to the use of the low methoxyl pectin of the invention for the manufacture of a product, wherein the product is selected from the group comprising food product, pharmaceutical product, personal care product, household product and consumer product.

Here, the food product is preferably selected from the group consisting of jam, marmalade, fruit preparation, jelly, dairy product and beverage.

In a fifth aspect, the invention relates to a product comprising the pectin according to the invention. Here, the product preferably comprises a polyvalent cation, which is particularly preferably calcium.

This pectin-containing product is preferably selected from the group consisting of food, pharmaceutical product, personal care product, household product and consumer product.

In a sixth aspect, the invention relates to a stabilized aqueous system comprising:
(i) at least one low methoxyl pectin according to the invention; and
(ii) at least one polyvalent cation.

In a preferred embodiment, the product or stabilized aqueous system described above is characterized in that the polyvalent cation is calcium.

In a further embodiment, the product or stabilized aqueous system described above is characterized in that the total amount of pectin is from 0.5 to 1.5 wt % per dry weight. Preferably, the total amount of pectin here is from 0.8 to 1.4 wt % per dry weight, and particularly preferably from 1.0 to 1.2 wt %.

Examples of pharmaceutical products in which the low methoxyl pectin of the present invention may be advantageously used include tablets, capsules, wound care products, and stoma products containing pectins in their formulations.

Examples of cosmetic products in which the low methoxyl pectin of the present invention may advantageously be used include products for cleansing, protection and care of the human or animal body which contain pectins in their formulations. They also include fragrance products and decorative cosmetics.

Examples of household products in which the low methoxyl pectin of the present invention may advantageously find use include air fresheners, cleaning products, and detergent formulations containing pectins in their formulations.

### Definitions

A “pectin” according to the application is defined as a plant polysaccharide which, as a polyuronide, consists essentially of α-1,4-glycosidically linked D-galacturonic acid units. The galacturonic acid units are partially esterified with methanol. The degree of esterification describes the percentage of carboxyl groups in the galacturonic acid units of pectin that are present in esterified form, e.g. as methyl esters.

A “low methoxyl pectin” according to the present invention has a degree of esterification of less than 50%. The degree of esterification is the percentage of carboxylic groups in the galacturonic acid chains of the pectin which are present in esterified form, e. g. as methyl esters. The degree of esterification can be determined with the method according to JECFA (Monograph 19-2016, Joint FAO/WHO Expert Committee on Food Additives).

“High methoxyl pectin” is defined as pectin with a degree of esterification of at least 50%. A low methoxyl pectin, on the other hand, has a degree of esterification of less than 50%. The degree of esterification is the percentage of carboxylic groups in the galacturonic acid chains of the pectin which are present in esterified form, e. g. as methyl esters. The degree of esterification can be determined with the method according to JECFA (Monograph 19-2016, Joint FAO/WHO Expert Committee on Food Additives).

The term “highly calcium-reactive pectin” is used here to refer to a pectin that has a calcium sensitivity of greater than 1500 HPE.

The term “fruit” in the context of the present invention refers to the entirety of the organs of a plant that emerge from a flower, including both the classical fruits and fruit vegetables. The term “fruit” in the sole position also includes mixtures of fruits of two or more different plants, such as apple tree and cherry tree, i.e. plant species, and/or mixtures of two or more different varieties of a fruit, for example two or more strawberry varieties, such as Donna®, Daroyal®, Lambada® and Symphony®. Analogous is true for expressions encompassing the term “fruit”, such as “fruit-containing” and “fruit preparation”.

“Food” in the context of the present invention means a food or feed.

A “food product” comprising one or more of the compositions described herein which comprise at least one low methoxyl gelling calcium pectinate and an ingredient selected, for example, from the group consisting of a fruit, a vegetable, cocoa, chocolate, a nut and a nut-like fruit, and mixtures thereof, is referred to herein as a “mixed food product”. A variety of food products of animal and/or plant origin may serve as the basis for the mixed food product. Food products of plant origin include, but are not limited to, milk substitute products.

“Milk substitute products” are in particular products containing water and a cereal variety, a pseudocereal variety, a legume variety, a nut variety, an almond variety, a nut-like fruit, or a mixture thereof. The term “pseudocereal” refers to all grain fruits of plants that do not belong to the genus of sweet grasses, i.e., cereals. Specifically, these are buckwheat, amaranth, quinoa and hemp.

As used herein, “plant-based milk substitute products” means products containing, for example, pea, soy, oat, spelt, millet, almond, hazelnut, coconut, cashew nut, rice, lupin seed, hemp seed, or a mixture thereof. The water contained therein may be derived from the respective plant ingredients used and/or may have been added during the manufacture of the respective plant-based milk substitute product. Furthermore, the plant-based milk substitute products may contain at least one added sugar and/or sugar substitute.

The term “sugar” also includes sugar substitutes, in particular sugar alcohols such as maltitol, sorbitol, mannitol, xylitol, isomalt, lactitol and erythritol, as well as inulin, isomaltulose, corn syrup, oligofructose, starch hydrolysate, trehalose and trehalulose.

At this point, it should be explicitly pointed out that features of the above mentioned solutions or in the claims described solutions can also be combined, if necessary, in order to be able to implement or achieve the explained features, effects and advantages in a correspondingly cumulative manner.

All features disclosed in the application documents are claimed to be essential to the invention, provided that they are, individually or in combination with each other, new compared to the prior art.

It should also be expressly pointed out that in the context of the present patent application indefinite articles and numerical indications such as “one”, “two”, etc. are as a rule to be understood as “at least” indications, i.e. as “at least one . . . ”, “at least two . . . ”, etc., unless it is expressly clear from the respective context or it is obvious or technically imperative for the person skilled in the art that only “exactly one . . . ”, “exactly two . . . ”, etc. can be meant there.

Further advantages, special features and expedient further embodiments of the invention are apparent from the subclaims and the following presentation of examples.

### Examples of Embodiment

1.1 Test Method for Determining the Degree of Esterification (DE)

This method corresponds to the JECFA (Joint FAO/WHO Expert Committee on Food Additives) published method. Deviating from the JECFA method, the de-ashed pectin is not dissolved in the cold, but heated. Isopropanol is used as alcohol instead of ethanol.

1.2 Test Method for Determining Calcium Sensitivity (CAE)

Materials:
320.0 g 0.65 M potassium acetate-lactic acid buffer solution (52.50 g potassium acetate; 271.25 g lactic acid make up to 5 liters with demineralized water)
60.0 g sugar (sucrose)
3.12 g pectin (equivalent to 0.82 wt % in the final product)
16.0 mL calcium chloride solution 5% (m/v)
Initial weight: approx. 399 g
Final weight: 380 g
Filling temperature: approx. 90° C.
pH value: approx. 3.0
Dry matter content: approx. 22%

Measurement method:
Mix pectin and the entire amount of sugar homogeneously in glass bowl.
Preheat electric heating plate on highest setting for at least 10 minutes.
Place buffer solution in a stainless steel saucepan.
Sprinkle the pectin-sugar mixture into the buffer solution while stirring, bring to boil and heat while stirring until the calcium pectinate is completely dissolved.
Dispense the calcium chloride solution and boil until final weight is reached.
At a temperature of about 90° C., quickly weigh 90 g of the boil into three test beakers with a tear FIGURE inserted and temper to 20° C. in a water bath.
Place the beakers in a water bath while avoiding shocks.
After exactly 2 h, the breaking strength is measured with a conventional pectinometer (e.g. Mark IV, Herbstreith & Fox, Neuenburg). The result is given as the mean value of the three individual values.

1.3 Determination of the Calcium Content (Complexometric)

Principle:

Calcium ions are titrated with Titriplex III (EDTA) at a pH between 12 and 13. Calconcarboxylic acid, which forms a red complex with calcium ions, is used as an indicator. During the titration, first the free calcium ions react with EDTA, then those bound to the indicator; the latter changes from red to blue in the process.

Recommended initial weight: Pectin: approx. 1-2 g

The accurately weighed sample (E) is ashed (2 h at 550° C. in a muffle furnace) and then dissolved in a small excess of dilute nitric acid (heated if necessary). The solution is quantitatively transferred to a 50 mL volumetric flask and filled with demineralized water (tempering in a water bath at 20° C.). An aliquot of this solution (a), which should contain 3-15 mg calcium, is pipetted into a wide-necked conical flask and this is made up with demineralized water to 150 mL. The pH is adjusted to 12-13 with 2 n NaOH (check pH) and approximately 0.1 g of calconcarboxylic acid indicator is added. The solution is titrated from red to pure blue with 0.025 m Titriplex III solution. The titration must be carried out immediately, otherwise calcium carbonate may precipitate as a result of absorption of CO₂ from the air. If the sample solution contains less than 3 mg calcium, it is recommended to use a 0.01 m Titriplex III solution for the titration. The consumption of 0.025 m or 0.01 m Titriplex III solution is noted (b).

Calculation:

Titration with 0.025 m Titriplex III Solution:

1 mL 0.025 m Titriplex III solution corresponds to 1.002 mg

$$\% ( m / m - ℊ / 100 ℊ ) Calcium = \frac{b * f * 1 , 002 * 50 * 100}{E * a * 1000}$$

Titration with 0.01 m Titriplex III Solution:

1 mL 0.01 m Titriplex III solution corresponds to 0.4008 mg calcium.

$$\% ( m / m - ℊ / 100 ℊ ) Calcium = \frac{b * f * 0 , 4008 * 50 * 100}{E * a * 1000}$$

E: sample quantity used for the analysis (ashing) in g
a: sample solution submitted for titration in mL
b: consumption of 0.025 m or 0.01 m Titriplex III solution in mL
f: factor of 0.025 m or 0.01 m Titriplex III solution

1.4 Determination of the Viscosity of a 2.5 wt % Pectin Solution
measuring device: Physica Rheolab MC 1/Rheolab QC
measuring system: Z2 DIN (disposable measuring cup, reusable measuring cup)
sample volume: 100 mL
sample treatment: The sample prepared by method 1.5 is tempered in a water bath at 20° C. for 15 minutes before measurement

Measuring Parameters:
1st stage (determining the viscosity) with the following settings:
default parameter: shearing speed [s⁻¹]
profile: ramp linear
value: 0.1-500 s⁻¹
measuring points: 21
measuring profile: constant measuring point duration
measuring point duration: approx. 5.71 s
section duration: 120 s
measured value generation: automatically
temperature: 20° C. (constant)

Evaluation:

The viscosity (unit: [mPas]) is read at a shear rate of 7=250 s^{-1.}

1.5 Preparation of a 2.5% Aqueous Solution of Pectin (Wt %)

Procedure:

Place boiling, demineralized water in a 250 ml beaker and sprinkle the sample directly into the stirring vortex while the stirrer is running. For complete dissolution, run the stirrer at the highest speed for approx. 1 minute.

Prepare an X wt % solution according to the following recipe:
Amount of sample: x g sample i.e. 2.5 g pectin
Amount of water: 100−x g water i.e. 97.5 g water

2. Preparation of a Low Methoxyl Pectin According to the Invention

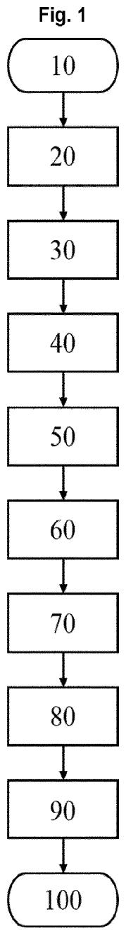

FIG. 1 shows a schematic flow diagram of a process for the production of low methoxyl pectin according to the invention. Starting from the citrus dry pomace 10, the pomace is subjected to hydrolysis 20 by incubation in an acidic medium. Here, the pomace is incubated by incubation in an aqueous solution at a pH between 1.0 and 3.0 for 2 to 6 hours, during which the protopectin is converted into soluble pectin. Subsequently, the pectin present in solution is separated from the pomace mass by means of a multi-stage solid-liquid separation 30 (e.g., by a decanter in the first step and a separator in the second step). In step 30, divalent ions are also removed by incubation with a cation exchanger such as Purolite A 100 Plus. This is followed by pH adjustment and enzymatic treatment of the aqueous pectin solution by addition of a pectin methyl esterase in step 50 (total activity 1000 to 2500 units/L) and incubation of the solution at 30 to 50° C. for 12 to 18 hours. In step 60, after falling below a critical pH limit, the pH is adjusted, by increasing the pH using a base such as NaOH or KOH. When the desired degree of esterification is reached, in step 70 the de-esterified pectin is precipitated by adding isopropanol and the precipitated pectin is separated from the reaction solution in step 80. Finally, in step 90, the pectin is gently dried by means of vacuum drying to then obtain the low methoxyl pectin 100 according to the invention.

3. Use of a Low Methoxyl Pectin Described Herein for the Preparation of Fruit Spreads

In one embodiment, a citrus pectin according to the invention, designated as pectin A, is used as a standardized pectin with minor addition of a high methoxyl pectin. The corresponding composition A is composed as follows:

| Ingredients | Amount | | Substance properties |
|---|---|---|---|
| Low methoxyl | 54 | wt % | Degree of esterification: 26.5% |
| citrus pectin | | | pH 4.5 |
| (pectin A) | | | Calcium sensitivity 1870 HPE |
| High methoxyl | 6 | wt % | Degree of esterification: 70.6% |
| citrus pectin | | | pH 4.5 |
| | | | Gelling strength: 241° USA-Sag |
| Saccharose | 40 | wt % | |

In a further embodiment, an apple pectin according to the invention designated as pectin B is used as a standardized pectin. The corresponding composition B is composed as follows:

| Ingredients | Amount | Substance properties |
|---|---|---|
| Low methoxyl | 60 wt % | Degree of esterification: 30.1% |
| apple pectin | | pH 3.76 |
| (pectin B) | | Calcium sensitivity 977 HPE |
| Saccharose | 40 wt % | |

Product formulation for an organic fruit spread with 30° Brix and 55% fruit content with composition A containing pectin A (see table above):

Recipe:

| | | |
|---|---|---|
| 240 | g | pectin solution 5% (=1.2%) with the composition |
| | | A described above |
| 550 | g | strawberries*, pureed (from organic cultivation) |
| 235 | g | sucrose*, crystalline (from organic cultivation) |
| 10 | g | lemon juice concentrate* 45° Brix |

Initial weight: approx. 1035 g
Final weight: approx. 1000 g
Dry matter: approx. 30%
pH values: approx. 3.3-3.5

Production:
A: Stir and dissolve pectin in hot demineralized water at 80° C. using a high-speed stirrer.
B: Mix pureed strawberries and sucrose and heat to approx. 90° C.
C: Add pectin solution “A” and heat again to 90° C.
D: Add lemon juice concentrate.
E: Filling temperature approx. 80-85° C.

Product formulation for an organic fruit spread with 30° Brix and 55% fruit content with composition B containing pectin B (see table above):

Recipe:

| | | |
|---|---|---|
| 240 | g | pectin solution 5% (=1.2%) with the composition |
| | | B described above |
| 550 | g | strawberries*, pureed (from organic cultivation) |
| 235 | g | sucrose*, crystalline (from organic cultivation) |
| 10 | g | lemon juice concentrate* 45° Brix |

Initial weight: approx. 1035 g
Final weight: approx. 1000 g
Dry matter: approx. 30%
pH values: approx. 3.3-3.5

Production:
A: Stir and dissolve pectin in hot demineralized water at 80° C. using a high-speed stirrer.
B: Mix pureed strawberries and sucrose and heat to approx. 90° C.
C: Add pectin solution “B” and heat again to 90° C.
D: Add lemon juice concentrate.
E: Filling temperature approx. 80-85° C.

4. Determination of the Breaking Strength of the Product Formulations Described in Point 3

Test Method for Determining the Breaking Strength:

After reaching the final weight, immediately weigh 100±1 g each of the boiling rapidly into three Luers beakers with inserted tear FIGURE.

Place the beakers in a water bath (20±1° C.) placed directly next to the boiling point, avoiding vibration, and temper. The Luers beakers must be in the water up to the filling level of the gel. The water level must be regulated when numerous specimens are placed in or removed from the water bath.

After exactly 20 hours, the breaking strength is measured with the Herbstreith Pectinometer Mark III or Mark IV (Herbstreith & Fox GmbH & Co. KG Pektin-Fabriken, Neuenburg, Germany). The result is the mean value of the three individual values.

Results:

The results are summarized in the following table:

| | | Breaking | | |
|---|---|---|---|---|
| | | strength | | |
| pectin | Dosage: | 20 h | pH | ° Brix |
| Pectin A (in the form of | 1.2% | 240 HPE | 3.34 | 30.0 |
| composition A) | | | | |
| Pectin B (in the form of | 1.2% | 320 HPE | 3.33 | 30.0 |
| composition B) | | | | |

Both fruit spreads showed high fracture strength with 240 and 320 HPE, respectively, even without addition of external calcium salts.

### LIST OF REFERENCE NUMBERS

10 citrus pulp
20 hydrolysis (disintegration) by incubation in an acidic environment
30 solid-liquid separation and partial de-ionization
40 concentrating and pH adjustment
50 addition of enzyme
60 pH adjustment
70 precipitation in alcohol
80 solid-liquid separation
90 drying
100 obtained low methoxyl, highly-calcium-reactive pectin

1. Method for the preparation of low methoxyl pectin comprising the following steps:
(a) Providing a pectin-containing starting biomass material that includes an insoluble fiber component and an insoluble protopectin component;
(b) Extraction of the pectin-containing starting biomass material with an aqueous medium at an acidic pH under conditions where at least a portion of the pectin content is extracted;
(c) Separation of the pectin from the treated starting biomass material and at least partial removal of divalent cations from the pectin extract;
(d) Concentrating the pectin extract from step (c) so that the pectin extract has a content of pectin-containing material as dry matter that is in the range of 0.5 to 10 wt %;
(e) Contacting the concentrated pectin extract from step (d) in an aqueous solution with a pectin methyl esterase (E.C. 3.1.1.11) at a pH of 3.8 to 4.5;
(f) Allow the pectin methylesterase to de-esterify the pectin to produce a de-esterified pectin by incubating the aqueous suspension from step (e);
(g) Terminating the de-esterification when the de-esterified pectin has a degree of esterification of from 10 to 34% and advantageously from 10 to 28.0%, and
(h) Obtaining low methoxyl pectin therefrom,
wherein in step (f) a further adjustment of the pH value is carried out by adding a pH-value-increasing substance or substance mixture.

2. Method for the preparation of low methoxyl pectin comprising the following steps:
(b1) Providing a pectin starting material in a dried state;
(c1) Preparing an aqueous solution of the pectin starting material from step (b1) such that the pectin-containing solution has a content of pectin-containing material as dry matter that is in the range of 0.5-10 wt %;
(e) Contacting the concentrated pectin-containing solution from step (c1) in an aqueous solution with a pectin methyl esterase (E.C. 3.1.1.11) at a pH of 3.8 to 4.5;
(f) Allow the pectin methylesterase to de-esterify the pectin to produce a de-esterified pectin by incubating the aqueous solution from step (e);
(g) Terminating the de-esterification when the de-esterified pectin has a degree of esterification of from 10 to 34% and advantageously from 10 to 28.0%, and
(h) Obtaining low methoxyl pectin therefrom,
wherein in step (f) a further adjustment of the pH value is carried out by adding a pH-value-increasing substance or substance mixture.

3. Method according to claim 1, characterized in that the pectin-containing starting biomass material or pectin starting material is obtained from a fruit selected from the group consisting of citrus, apple, sugar beet, infructescences of sunflower, rosehip, quince, apricot, cherry, carrot, and mixtures thereof.

4. Method according to claim 1, characterized in that the pectin-containing starting biomass material is in a moist, non-dried state, a frozen state or a dried state.

5. Method according to claim 4, characterized in that the pectin-containing starting biomass material is citrus pomace or apple pomace.

6. Method according to claim 1, characterized in that the extraction in step (b) satisfies one or more of the following conditions:
i. Use of an organic acid such as oxalic acid or citric acid;
ii. Use of a mineral acid such as sulfuric acid, hydrochloric acid, nitric acid or sulfurous acid, wherein nitric acid or sulfuric acid is preferred;
iii. The pH of the suspension is between pH=0.5 and pH=4.0, preferably between pH=1.0 and pH=3.5, and particularly preferably between pH=1.0 and pH=3.0;
iv. The incubation is carried out at a temperature between 60° C. and 95° C., preferably between 70° C. and 90° C. and particularly preferably between 75° C. and 85° C.;
v. The incubation is carried out for a period of time between 60 min and 8 h, preferably between 2 h and 6 h;
vi. The suspension has a dry matter of between 0.5% and 5%, preferably between 1% and 4%, and more preferably between 1.5% and 3%;
vii. The suspension is set in motion by the application of force during disintegration, preferably by stirring or shaking.

7. Method according to claim 1, characterized in that the separation of the pectin from the treated starting material in step (c) satisfies one or more of the following conditions:
i. The separation is carried out as a single-stage or multi-stage separation;
ii. The separation is performed using a device selected from the group consisting of a decanter, separator, wet press, belt press, or sieving machine;
iii. The separation involves as complete a separation of particles as possible.

8. Method according to claim 1, characterized in that the removal of divalent ions from the pectin extract in step (c) satisfies one or more of the following conditions:
i. A cation exchanger material is used that is either implemented in a batch process or a column process;
ii. A chelator is used to remove the divalent ions;
iii. Removal of the divalent ions is accomplished with precipitation as a poorly soluble salt.

9. Method according to claim 1, characterized in that the enzymatic de-esterification in step (f) satisfies one or more of the following conditions:
i. At least one pectin methyl esterase (EC 3.1.1.11) is added to the aqueous solution;
ii. The aqueous solution contains pectin methyl esterase in a total activity of from 100 to 10000 units/L, advantageously from 500 to 5000 units/L, and particularly advantageously from 1000 to 2500 units/L;
iii. The incubation with the at least one pectin methylesterase in the aqueous solution is carried out for a period of time of from 10 to 20 hours, and preferably from 12 to 18 hours;
iv. The incubation with the at least one pectin methyl esterase is carried out at a temperature of between 10° C. and 70° C., preferably of between 20° C. and 60° C. and more preferably of between 30° C. and 50° C.;
v. The incubation with the at least one pectin methyl esterase is carried out at an initial pH of between 3.8 to 4.5, preferably of between 4.0 to 4.3 and more preferably of between 4.1 to 4.2;
vi. The dry matter in the aqueous solution is between 0.5 wt % and 6 wt %, preferably between 1 wt % and 4 wt %, and more preferably between 2 wt % and 3 wt %;
vii. The aqueous solution is set in motion during incubation by application of force.

10. Method according to claim 1, characterized in that the adjustment of the pH in step (f) is a stepwise pH increase, which is carried out by discrete addition of a pH-value-increasing substance or substance mixture.

11. Method according to claim 10, characterized in that the adjustment involves the discrete addition of the pH-value-increasing substance or mixture of substances at an incubation time of 1 to 6 h or when a pH of between 3.5 and 4.5 is reached.

12. Method according to claim 1, characterized in that the adjustment of the pH value in step (f) is a pH value maintenance which is carried out by continuous addition of a pH-value-increasing substance or substance mixture.

13. Method according to claim 1, any of the above 1 m characterized in that the pH-value-increasing substance is a base which is preferably present as a mixture of substances in aqueous solution.

14. Method according to claim 13, characterized in that the base is selected from the group consisting of ammonia, sodium hydroxide or potassium hydroxide.

15. Method according to claim 1, characterized in that the pH-value-increasing substance is a slightly acidic or basic salt which is preferably present as a mixture of substances in aqueous solution.

16. Method according to claim 15, characterized in that the slightly acidic or basic salt is selected from the group consisting of potassium carbonate, sodium carbonate, sodium hydrogen carbonate and potassium hydrogen carbonate.

17. Method according to claim 1, characterized in that the pH-value-increasing substance mixture is a slightly acidic or basic buffer system.

18. Method according to claim 17, characterized in that the slightly acidic or basic buffer system is selected from the group consisting of carbonic acid/carbonate buffer, acetic acid/acetate buffer, phosphate buffer, ammonia buffer, HEPES buffer, PBS buffer and MES buffer.

19. Low methoxyl pectin obtainable or obtained by a process according to claim 1, characterized in that it has a degree of esterification of from 10 to 34% and advantageously from 10 to 28.0%.

20. Low methoxyl pectin according to claim 19, characterized in that it has a calcium sensitivity of 300 HPE to 3000 HPE.

21. Use of a low methoxyl pectin according to claim 19 for the manufacture of a product, wherein the product is selected from the group comprising food product, pharmaceutical product, personal care product, household product and consumer product.

22. Use according to claim 21, characterized in that the food is selected from the group comprising jam, marmalade, fruit preparation, jelly, dairy product and beverage.

23. Product comprising a pectin according to claim 19, wherein the product preferably comprises a polyvalent cation.

24. A stabilized aqueous system comprising:
(i) at least one low methoxyl pectin according to claim 19; and
(ii) at least one polyvalent cation.

25. Product according to claim 23 or stabilized aqueous system according to claim 24, characterized in that the polyvalent cation is calcium.

26. Product according to claim 23 or stabilized aqueous system according to claim 24 or 25, characterized in that the total amount of pectin is from 0.5 to 1.5 wt % per dry weight, preferably from 0.8 to 1.4 wt % per dry weight and more preferably from 1.0 to 1.2 wt % per dry weight.
